(19) United States
(12) Patent Application Publication
(10) Pub. No.: US 2008/0097223 A1
(43) Pub. Date: Apr. 24, 2008

(54) Optical Catheter Carriage Interlock System and Method

(75) Inventors: Peter Strickler, Tewksbury, MA (US); John Murphy, North Reading, MA (US)

Correspondence Address: HOUSTON ELISEEVA, 4 MILITIA DRIVE, SUITE 4, LEXINGTON, MA, 02421, US

(73) Assignee: INFRAREDX, INC., Burlington, MA (US)

(21) Appl. No.: 11/875,590

(22) Filed: Oct. 19, 2007

Related U.S. Application Data
(60) Provisional application No. 60/862,309, filed on Oct. 20, 2006.

(51) Int. Cl. A61B 6/00

(52) U.S. Cl. 600/478; 604/21; 606/15

(57) ABSTRACT

An optical catheter system comprising an intraluminal catheter that provides optical signals to a patient and carries optical signals from the patient, an outer housing, and an inner carriage that moves longitudinally relative to the outer housing and rotates relative to the outer housing during operation when the catheter system is being driven by a pullback and rotation system. The optical catheter system has an interlock system that prevents rotation and longitudinal movement of the inner carriage in the outer housing until attached to the pullback and rotation system. The pullback and rotation system comprises a frame and a catheter system interface, attached to the frame, to which the catheter system is coupled. A carriage drive system is further provided that moves longitudinally and rotates relative to the frame to provide rotation and longitudinal drive to the catheter system. A longitudinal drive system has a drive motor for advancing and/or withdrawing the carriage drive system and a manual drive input enabling a user to manually advance or withdrawal the carriage drive system. A latching system holds the carriage drive system when the catheter system is being attached to the pullback system.

### RELATED APPLICATIONS

This application claims the benefit under 35 USC 119(e) of U.S. Provisional Application No. 60/862,309, filed on Oct. 20, 2006 and is related to U.S. application Ser. No. ______, entitled “Manual and Motor Driven Optical Pullback and Rotation System and Method,” by John Murphy and Peter Strickler, Attorney Docket No. 0010.0013US2, filed on even date herewith, U.S. application Ser. No. ______, entitled “Pullback Carriage Interlock System and Method for Catheter System,” by John Murphy and Peter Strickler, Attorney Docket No. 0010.0013US3, filed on even date herewith and U.S. application Ser. No. ______, entitled “Noise Suppression System and Method in Catheter Pullback and Rotation System,” by Charles Abele and Jay Caplan, Attorney Docket No. 0010.0013US4, filed on even date herewith, all four of which are incorporated herein by reference in their entirety.

### BACKGROUND OF THE INVENTION

Catheter-based optical systems are applicable to a number of diagnostic and therapeutic medical applications. Optical tomography, usually optical coherence tomography (OCT), is used to provide spatial resolution, enabling the imaging of internal structures. Spectroscopy is used to characterize the composition of structures, enabling the diagnosis of medical conditions by differentiating between cancerous, dysplastic, and normal tissue structures, for example. Reflectance analysis is a simplified form of spectroscopy that analyzes optical properties of structures, typically in specified wavelength bands. Fluorescence and Raman spectral analysis involve exciting the tissue at one wavelength and then analyzing light at fluorescence wavelengths or Raman shifted wavelengths due to a process of inelastic photon scattering. They all share certain catheter requirements including the need to transmit an optical signal to the internal structures of interest and then detect returning light, often transmitting that returning light back along the length of the catheter.

For example, in one specific spectroscopic application, an optical source, such as a tunable laser, is used to access or scan a spectral band of interest, such as a scan band in the near infrared wavelengths or 750 nanometers (nm) to 2.5 micrometers (μm) or one or more subbands. The generated light is used to illuminate tissue in a target area in vivo using the catheter. Diffusely reflected light resulting from the illumination is then collected and transmitted to a detector system, where a spectral response is resolved. The response is used to assess the composition and consequently the state of the tissue.

This system can be used to diagnose atherosclerosis, and specifically to identify atherosclerotic lesions or plaques. This is an arterial disorder involving the intimae of medium- or large-sized arteries, often including the aortic, carotid, coronary, and cerebral arteries.

Diagnostic systems including Raman and fluorescence-based schemes have also been proposed. Other wavelengths, such as visible or the ultraviolet, can also be used.

In OCT applications, a coherent optical source is used to illuminate tissue in a target area. By analysis of the interference between light returning from the target area and light returning from a reference arm, depth information is generated providing information of both the surface topology and subsurface structures.

Other, non-optical, technologies also exist. For example, intravascular ultrasound (IVUS) uses a combination of a heart ultrasound (echocardiogram) and cardiac catheterization. In this application, an ultrasound catheter is inserted into an artery and moved to a target area. It then both generates and receives ultrasound waves that can then be constructed into an image showing the surface topology and internal structures at the target area.

The probes or catheters for these applications typically have small lateral dimensions. This characteristic allows them to be inserted into incisions or lumen, such as blood vessels, with lower impact or trauma to the patient. The probe's primary function is to convey light to and/or receive light from a target area or area of interest in the patient for the optical-based technologies. In the context of the diagnosis of atherosclerosis, for example, the target areas are regions of the patient's arteries that may exhibit or are at risk for developing atherosclerotic lesions.

In each of these applications, the target areas or areas of interest are typically located lateral to the catheter head. That is, in the example of lumens, the probe is advanced through the lumen until it reaches the areas of interest, which are typically the lumen walls that are adjacent to the probe, i.e., extending parallel to the direction of advance of the probe. A “side-firing” catheter head emits and/or receives light or ultrasound signals from along the probe's lateral sides. In the example of catheters for optical-based applications, the light propagates through the probe, until it reaches the probe or catheter head. The light is then redirected to be emitted radially or in a direction that is orthogonal to the direction of advancement or longitudinal axis of the probe. In the case of light collection, light from along the probe's lateral sides is collected and then transmitted through the probe to an analyzer where, in the example of spectroscopic analysis in the diagnosis of atherosclerosis, the spectrum of the returning light is resolved in order to determine the composition of the vessel or lumen walls.

In order to fully characterize target areas, relatively long regions of tissue, such as blood vessels, must be scanned and in the case of blood vessels an entire 360 degree circumference of vessels must be captured. To perform this combination of longitudinal and rotational movement, the catheters are typically driven by a device called a pullback and rotation (PBR) system.

Pullback and rotation systems connect to the proximal end of the catheter. They typically hold an outer sheath or jacket stationary while an inner catheter scanning body, including the catheter head are rotated and withdrawn through a segment of the blood vessel. This scanning combined with driving the catheter head produce a helical scan that is used to create a raster-scanned image of the inner walls of the blood vessel.

### SUMMARY OF THE INVENTION

In general, according to one aspect, the invention features a catheter system, comprising an intraluminal catheter for insertion into a patient and a handle portion. The intraluminal catheter includes an outer jacket and an inner scanning body that rotates and moves longitudinally within the outer jacket. The handle portion includes an outer housing mechanically coupled to the outer jacket and an inner carriage mechanically coupled to the inner scanning body.

This combination provides pullback and rotation scanning when the inner carriage is extracted and driven by a pullback and rotation system but ensures that the system is robust during transportation or when otherwise not being used.

This basic configuration can be used for optical catheters or catheters using other analysis modalities, such as IVUS. In the example of optical catheters, systems using spectroscopic, optical tomography, Raman, and fluorescence analysis modalities, for example, are compatible with this basic design.

In a preferred embodiment, the inner scanning body comprises an optical fiber bundle, including at least one optical fiber, for transmitting optical signals between a head of the intraluminal catheter and the inner carriage. In this case, the inner carriage preferably comprises one or more optical connectors for providing optical connection to the optical fiber bundle.

A torque cable is preferably provided for transferring rotation from the inner carriage through the inner scanning body to a head of the intraluminal catheter.

Typically, the outer housing functions as a handle for attachment of the catheter system to the pullback and rotation system. To facilitate alignment, the inner carriage is provided with a bayonet member projecting axially from a proximal side of the inner carriage for rotationally aligning the inner carriage during attachment to a pullback and rotation system.

A catheter carriage interlock system is preferably used to secure the inner carriage within the outer housing at least during transportation or when otherwise not in use. This catheter carriage interlock system prevents rotation of the inner carriage within the outer housing and extraction of the inner carriage from the outer housing.

In the preferred embodiment, a release member on a pullback and rotation system disengages the catheter carriage interlock system so that the inner carriage is able to rotate and move relative to the outer housing.

In general, according to another aspect, the invention features a catheter carriage interlock system for an optical catheter system comprising an intraluminal catheter that provides optical signals to a patient and carries optical signals from the patient, an outer housing, and an inner carriage that moves longitudinally relative to the outer housing and rotates relative to the outer housing during operation when the catheter system is driven by a pullback and rotation system. The interlock system comprises a carriage locking system that prevents rotation and longitudinal movement of the inner carriage in the outer housing. An unlocking system on the pullback and rotation system unlocks the carriage locking system to free the inner carriage to rotate and move longitudinally in the outer housing when the catheter system is connected to the pullback and rotation system.

In general, according to still another aspect, the invention features a carriage interlock method, comprising preventing rotation and longitudinal movement of the inner carriage in the outer housing at least during transportation of the optical catheter system and unlocking the inner carriage to free the inner carriage to rotate and move longitudinally in the outer housing when the optical catheter system is connected to a pullback and rotation system.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a side-plan view showing a catheter system according to the present invention;

FIG. 2 is a side cross-sectional view of the catheter system;

FIG. 3 is a side cross-sectional view of the catheter system showing the carriage interlock system shown in an open condition;

FIG. 4 is a perspective side view of a pullback and rotation system according to the present invention;

FIG. 5 is a partial side perspective view showing the axial drive system for the pullback and rotation system;

FIG. 5A is a schematic view showing the axial drive system for the carriage drive system;

FIG. 6 is a partial front perspective view of the carriage drive system of the pullback and rotation system;

FIG. 7 is a partial reverse angle perspective view of the carriage drive system for the pullback and rotation system;

FIG. 7A is a block schematic plan showing the optical path for the catheter and pullback and rotation system of the present invention;

FIG. 8 is a partial side perspective view of the pullback and rotation system showing the carriage drive locking system;

FIG. 9 is a partial side perspective view of the carriage drive locking system of the present invention;

FIG. 10 is a front plan view showing the catheter locking system; and

FIG. 11 is a front perspective view of the catheter locking system according to the present invention.

### DETAILED DESCRIPTION OF THE PREFERRED EMBODIMENTS

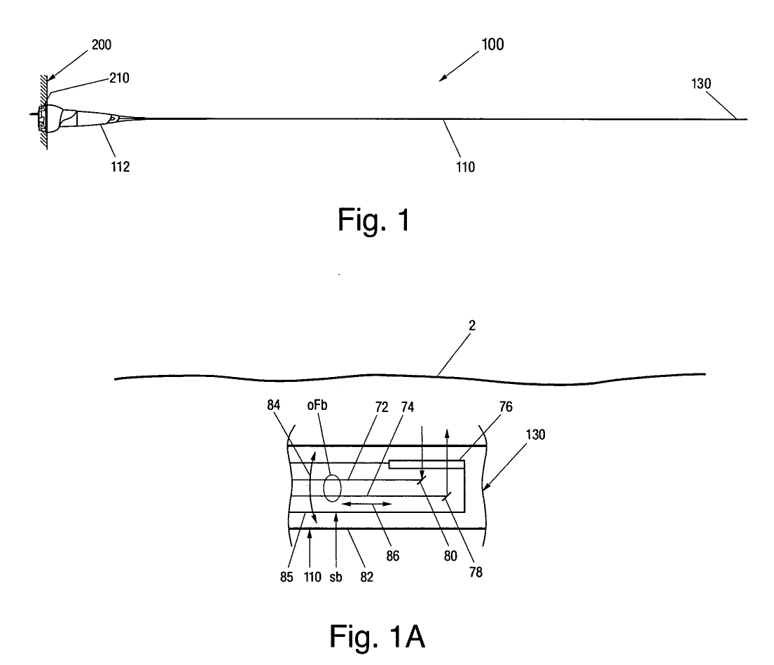

FIG. 1 shows a catheter system 100 connected to a pullback and rotation system 200, which have been constructed according to the principles of the present invention.

Generally, the catheter system 100 comprises an intraluminal catheter 110. This is typically inserted into a lumen within a patient, such as a blood vessel, particularly an artery. It is moved through the arterial network of the patient until a catheter head 130 is proximal or adjacent to a region of interest, such as potential site of a lesion within the coronary or carotid artery, for example.

FIG. 1A shows the intraluminal catheter 110 comprising an outer jacket 82 and an inner catheter scanning body sb including the catheter head 130. In operation, optical signals, such as a tunable signal that is spectrally scanned or tuned over a spectral scan band or a broadband optical signal, are transmitted to the head on a delivery fiber 74 of an optical fiber bundle ofb of inner catheter scanning body sb. The optical signal of the delivery fiber is directed to exit from the side of the head 130 by an angle reflector 78 through a window 76. Returning light, such as scattered and diffusely reflected light from the region of interest of the inner luminal walls 2 is captured by collection reflector 80 to be transmitted in a collection fiber 72.

In other examples, the delivery fiber transmits an excitation optical signal for Raman or fluorescence analysis. A narrowband optical signal is often used in reflectance analysis systems.

In order to enable scanning of the inner luminal walls 2, inner catheter scanning body sb including the head 130 is rotated within a protective jacket or sheath 82, see arrow 84, while typically being simultaneously translated longitudinally within the jacket 82, see arrow 86. The scanning body typically comprises an outer torque cable 85 for transferring rotation to the head 130. In the current embodiment, the torque cable 85 comprises contrahelically wound wire layers to enable low backlash torque transfer along the length of the intraluminal catheter 110. The jacket 82 ensures that the lumen is not damaged by the rotation 84 and longitudinal movement 86 of the inner catheter scanning body sb.

Returning to FIG. 1, the proximal end of the catheter system 100 has a catheter handle housing 112. This housing 112 is typically the portion of the catheter system 100 that is held by the medical personnel during some operations such as when attaching the catheter system 100 to a pullback and rotation system 200.

The pullback and rotation system 200 controls the movement of the inner catheter scanning body sb and catheter head 130 both in terms of rotation 84 and longitudinal movement 86 to typically helically raster scan the internal walls 2 of the coronary artery, for example, to assess and characterize any tissue, lesions, or other problems in and on those internal walls 2.

In other examples, the catheter and head are configured for OCT analysis. In still other examples, the catheter and head are used for IVUS applications. As such, the optical components are replaced or augmented by ultrasonic transducers in the head 130, for example.

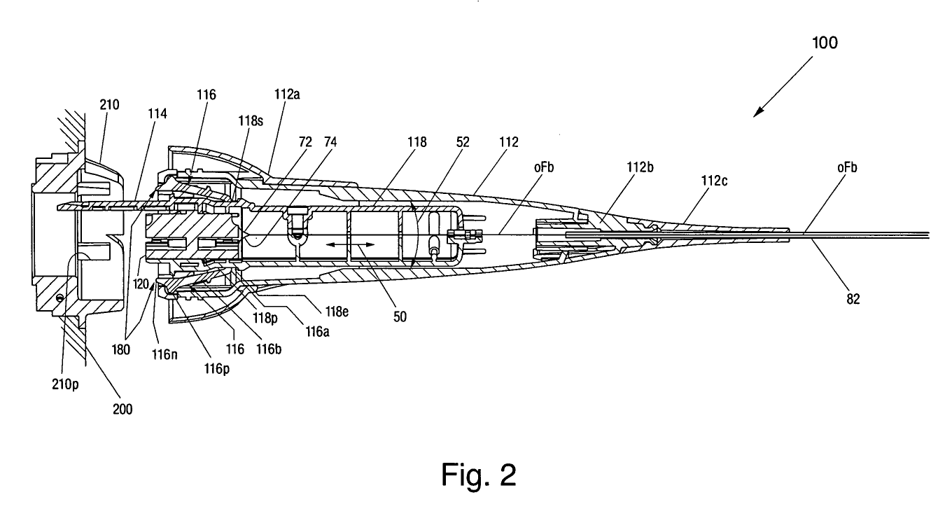

FIG. 2 shows the proximal end of the catheter system 100. It comprises the handle housing 112, providing a sterile field surrounding the internal components of the catheter system 100. The handle housing 112 further comprises a housing apron 112a that flares moving proximally in order to protect the coupling components housed within the housing 112. In contrast, moving distally, the housing further comprises a jacket fixing block 112b. The catheter jacket 82 is rigidly bonded to the jacket fixing block 112b such that the jacket 82 is stationary with respect to the housing 112, ensuring that the inner catheter scanning body sb moves with respect to the jacket 82. Finally, the distal end of the housing comprises a flexible nose portion 112c to prevent crimping of the catheter.

Within the housing 112 is a catheter carriage 118. The optical fiber bundle ofb is secured to the carriage 118 so that rotation 52 of the carriage or longitudinal movement 50 is transferred to the catheter head 130. The optical fiber bundle ofb in one embodiment, comprises the delivery fiber 74, which in one example is single spatial mode fiber that transmits an optical spectroscopy signal, such as a tunable signal generated by a tunable laser, to the catheter head 130, and the collection fiber 72, which is often multimode fiber, that transmits any collected light by the catheter head 130 through the length of the catheter system 100.

The catheter system 100 has a series of components that form a catheter carriage interlock system 180, which prevents the carriage 118 from moving within the housing body 112 both rotationally and longitudinally 50 when the catheter system 100 is not mechanically connected to the pullback and rotation system 200. However, an unlocking or key system on the pullback and rotation system 200 unlocks the carriage interlock system 180 to free the inner carriage 118 to rotate and move longitudinally in the housing 112 when the catheter system 100 is connected to the pullback and rotation system 200.

The interlock system 180 comprises a series of catheter locking levers 116, that prevent the carriage 118 from rotating 52 and being extracted from the housing 112 when the catheter system 100 is not connected to the pullback and rotation system 200 yet allow the carriage 118 to rotate within the housing body 112 and to move axially out of the body 112 when the catheter system 100 is connected to the pullback and rotation system 200. Specifically, in one example, more than two locking levers are used, such as four in one implementation.

Each catheter locking lever comprises a lever pivot 116p, a ring engagement nose 116n, and a lever arm 116a. When the catheter system 100 is not connected to the pullback and rotation system 200, the lever arms 116a of the catheter locking levers 116 are in engagement with an outer periphery 118p of the carriage 118. This prevents the rotation of the carriage 118 within the housing body because of the interference between the lever arm 116a and carriage rotation shoulders 118 of the carriage 118. Specifically, when the carriage 118 is fully inserted into the body, the lever arms 116a are resiliently biased against the catheter carriage 118 at region 118p and fall between adjacent, axially-extending carriage rotation shoulders 118s and thereby prevent the catheter carriage 118 from rotating within the housing body 112.

The resilient biasing of the lever arms 116 is provided by a flexible circular band 116b that extends around the outer periphery of the array of lever arms 116. In a current embodiment, the band 116b is fabricated from a synthetic rubber material such as EPDM (ethylene propylene diene monomer) rubber. This is a low creep, sterilization resistant material. In other implementations, the resilient biasing is performed by spring elements, such as leaf springs, that are integrally formed with the lever arms 116.

The engagement of the lever arms 116a against region 118p of the catheter carriage 118 also prevents the catheter carriage 118 from being extracted from the housing body 112. Specifically, if an extraction force is applied to the catheter carriage 118 relative to the housing body 112, the lever arms 116a slide along portion 118p of the catheter carriage 118 to engage with the extraction shoulder 118e. This mechanical interference thus prevents the catheter carriage 118 from being extracted from the housing body 112 or falling out when the catheter system 100 is not coupled to the pullback and rotation system 200.

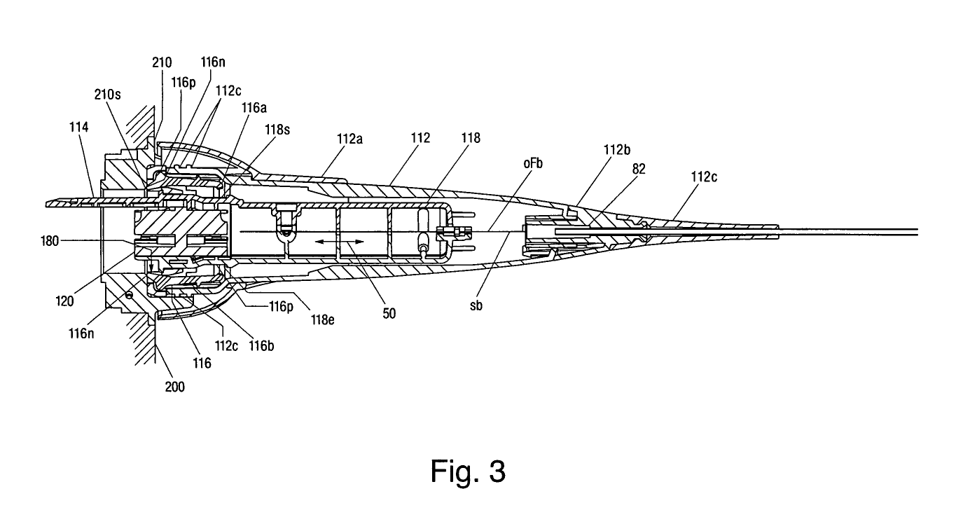

FIG. 3 shows the catheter system 100 coupled to the pullback and rotation system 200 and specifically its interaction with the interface release ring 210. The ring shoulders 210s engage with the ring engagement noses 116n of the catheter locking levers 116. This causes the locking levers 116 to pivot on the respective lever pivot 116p against the axially inward directed bias force of the band 116b with the lever arm portions 116a of the locking levers 116 rotating outward thereby bringing the lever arms 116a out of engagement with region 118p of the catheter carriage 118. This allows the catheter carriage 118 to now rotate within the housing 112 because the lever arms 116a are no longer interfering with the carriage rotation shoulders 118s. Further, the lever arms 116a are now pulled away from the carriage extraction shoulders 118e to thereby allow the carriage to move in the direction of arrow 10 and rotate in the direction of arrow 50′ relative to the housing body 112.

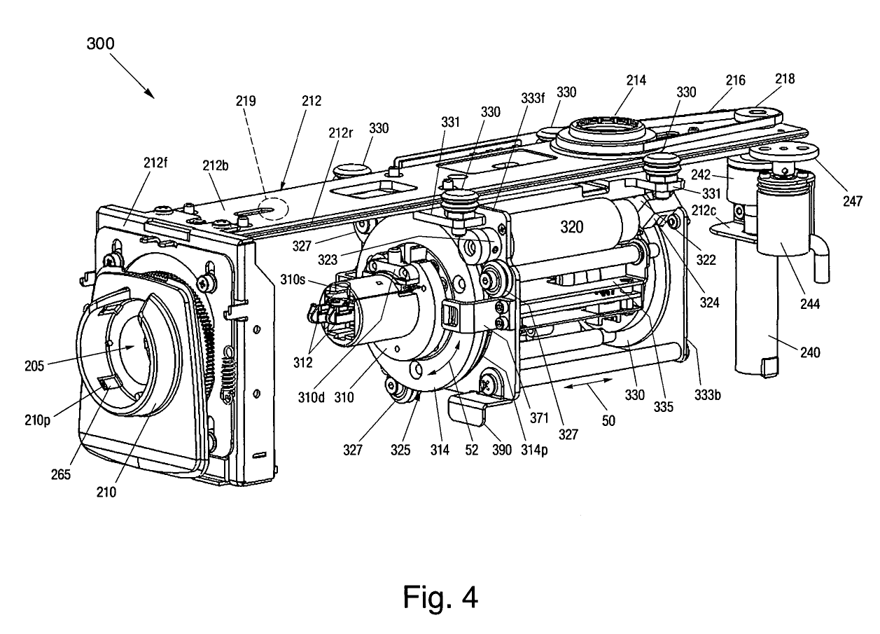

FIG. 4 shows the pullback and rotation system 200. It generally comprises a pullback and rotation frame 212. A front member 212f of the frame 212 holds the interface release ring 210 that forms part of the catheter interface 205 to which the catheter system 100 connects. A center member 212b runs laterally from the front member 212f to a rear member 212c.

The pullback and rotation system 200 also comprises a carriage drive system 300 that couples to the catheter carriage 118. This carriage drive system 300 generally drives the rotation of the inner catheter scanning body sb and the catheter head 130 of the catheter system 100 via the catheter carriage 118 and also drives the movement of the inner catheter scanning body sb and the catheter head 130 longitudinally in the catheter system 100. The longitudinal movement is provided by the movement of the carriage drive system 300 back and forth in the direction of arrow 50 and the rotation is accomplished by the rotation of a drum system 325 of the carriage drive system 300 in the direction of arrow 52.

In more detail, the carriage drive system 300 travels longitudinally on the pullback and rotation frame 212 on frame rails 212r formed on either side of the center member 212b. Specifically, carriage rollers 330 roll on the rails 212r thereby allowing the carriage drive system 300 to move laterally on the frame 212. The carriage rollers 30 are journaled to roller plates 331 which are attached to a front carriage frame plate 333f and a back carriage frame plate 333b, respectively.

The carriage drum system 325 is mounted to rotate on the front carriage frame plate 333f and the back carriage frame plate 333b. Specifically, the carriage drum system 325 comprises a front carriage drum roller 314 and a rear carriage drum base 330. Optical/electronic boards 335 extend between the drum base 330 and drum roller 314 and contain the electronic, optical, and opto-electronic components of the rotating drum system 325. The front carriage drum roller 314 supports a carriage coupler mount 310. The carriage coupler mount 310 holds a male optical duplex coupler 312 that connects to the female duplex optical coupler 120 of the catheter system 100. Specifically, this provides the optical connection between a delivery channel provided by delivery fiber 74 and collection channel provided by the collection fiber 72 of the optical fiber bundle ofb. A catheter alignment bayonet 114 projects proximally from the female duplex optical coupler 120.

The carriage coupler mount 310 also has a bayonet scabbard 310s that is a port for receiving the catheter alignment bayonet 114. Thus, upon insertion of the catheter system 100 into the pullback and rotation system 200, the catheter alignment bayonet 114 extends into the bayonet scabbard 310s to insure that the catheter system 100 and specifically the catheter carriage 118 is rotationally aligned to the drum system 325 of the carriage system 300 thus ensuring alignment between the female duplex optical coupler 120 and the male optical duplex coupler 312.

Further, the carriage coupler mount 310 further comprises a bayonet presence detector 310d that senses the presence of the catheter alignment bayonet 114 to thereby signal to the PBR system 200 when the catheter system 100 is properly connected to the PBR system.

The drum system 325 rotates relative to the carriage frame plates 333f, 333b under power of a carriage motor encoder 320. Specifically, the carriage motor encoder 320 drives a roller 323 that engages teeth on the outer periphery of the front drum 314. Thus, the motor encoder 320 drives the drum system 323 to rotate 52 under angular control of its encoder. Three carriage rollers 327, each having a female V-shape profile, provide support to the drum 325 by engaging a V-shaped outer periphery 314p of the front drum 314 at three distributed points of contact allowing its rotation.

The carriage drive system 200 also comprises a drum angular position detection system. Specifically, an angular position detector 324 is attached to the back carriage frame 333b of the carriage frame. The drum base 330 further comprises a flag 322 that passes in proximity to the angular position detector 324 and in this way the angular position of the drum system 325 in the carriage drive system 300 is detected and specifically its proper orientation to receive the catheter system 100 and in the alternative used to calibrate the encoder of the motor encoder 320 to a known reference.

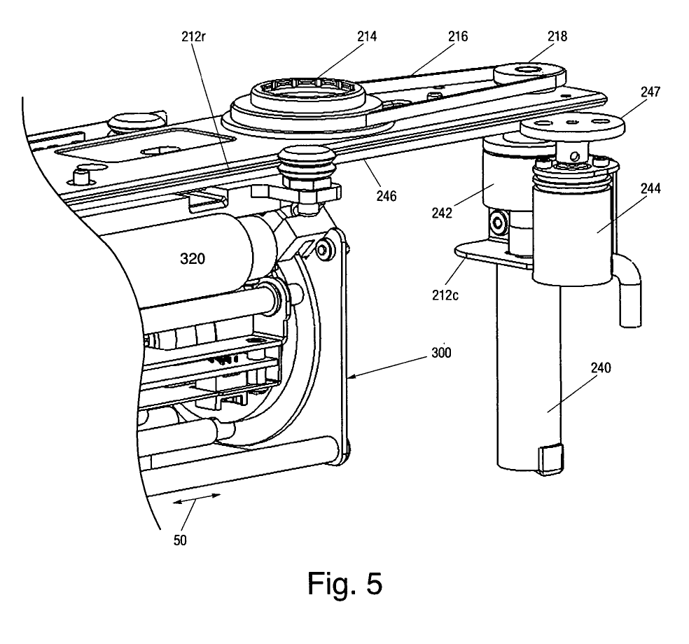

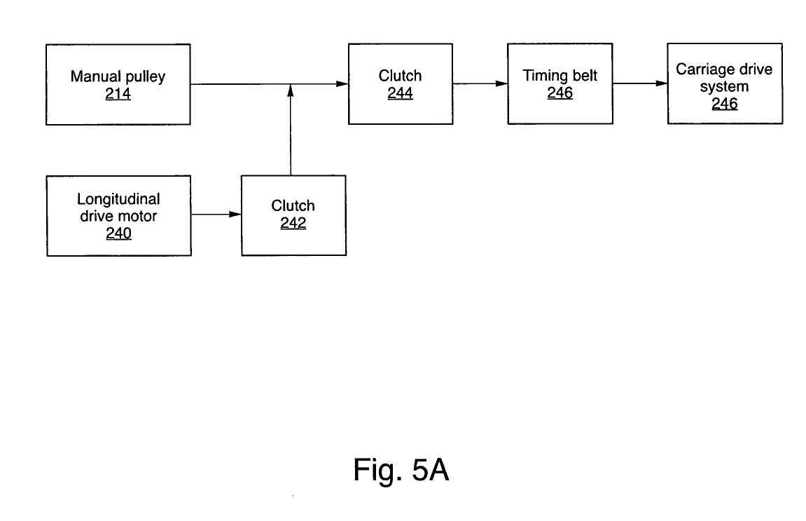

FIG. 5 shows the longitudinal drive system for the carriage drive system 300. The longitudinal drive system provides for movement both under direct operator control and under motor control. The manual operation, i.e., longitudinal movement, arrow 50, of the carriage drive system 300 is accomplished by user rotation of the manual pulley 214. This drives the manual drive belt 216 that turns the manual belt pulley 218. This movement causes the carriage drive system 300 to move back and forth in the direction of arrow 50 depending on the direction that the manual pulley 214 is rotated by the operator. In more detail, a timing belt drive belt 246 stretches between a pulley below the manual belt pulley 218 to a second timing belt pulley (see 219 in FIG. 4). The timing belt 246 is further attached to the carriage drive system 300. A longitudinal drive or timing belt drive motor 240, hung on the rear member 212c, is also alternatively used to drive the carriage drive system 300 back and forth in the direction of arrow 50. Specifically, the longitudinal drive motor 240 engages the timing belt 246 via a clutch 242. Thus, when the clutch 242 is engaged, the drive motor 240 connects to drive the carriage drive system 300 longitudinally. An encoder 244, attached to the rear member 212c, is also provided on the drive path, specifically the encoder 244 engages the timing belt 246 via an encoder pulley 247, specifically engaging the outer periphery of the timing belt 246 to monitor the axial position of the carriage system 300 on the rails 212r.

FIG. 5A schematically shows the mechanical system for driving the carriage drive system 300. Specifically, it allows for the encoder 244 to monitor the axial position of the carriage system 300 via the timing belt 246 regardless of whether the linear drive for the carriage drive system 300 is being provided manually, by the operator using manual pulley 214, or under control of the longitudinal drive motor 240. Specifically, the manual pulley 214 and potentially the drive motor 240 both drive the timing belt 246 that goes to the encoder 244. Thus, independent of the status of the clutch 242, being open or closed, the encoder 244 continues to monitor the position of the carriage drive system 300.

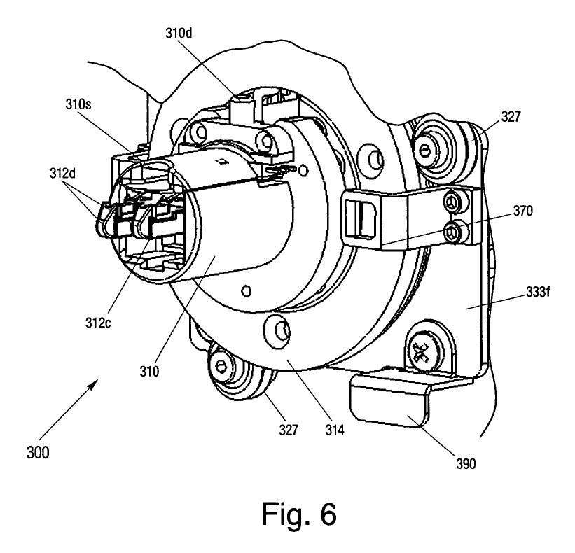

FIG. 6 is a close-up view showing the male duplex optical couplers 312. Specifically, the couplers are housed within the carriage coupler mount 310. Two optical male adapters, one for the multimode collection fiber (312c) and one for the single mode delivery fiber (312d) are provided. Each adapter has a front dust cover 312d that is closed when the connectors are not engaged to thereby protect the sensitive optical fiber end facets within the couplers. Presently, the Diamond-brand F-3000 Backplane adapters are used, which provide active push pull retention.

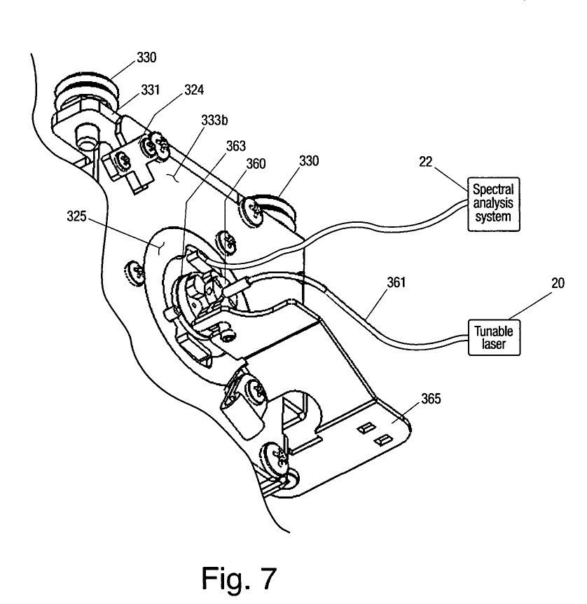

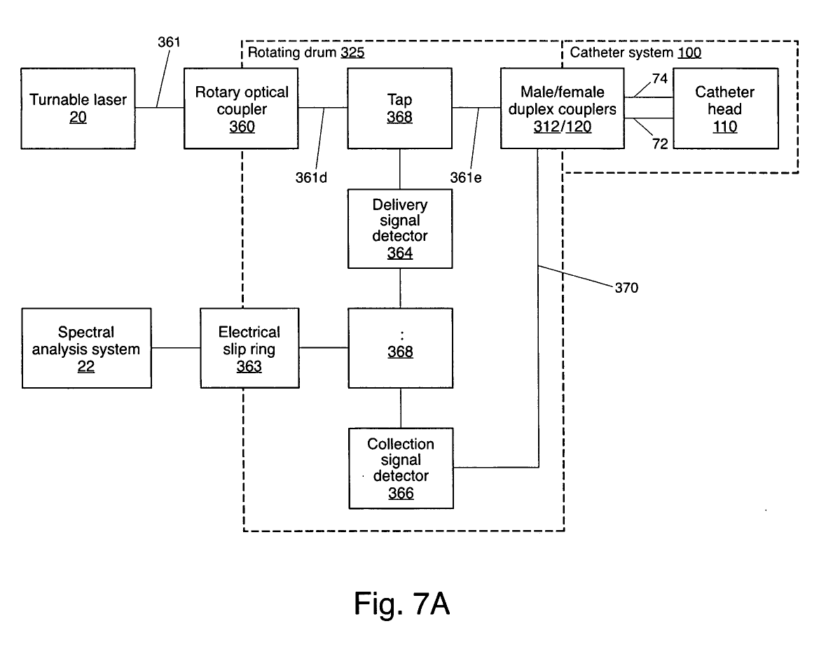

FIG. 7 is a more detailed partial view in a reverse angle better showing the optical and electrical connections for the carriage drive system 300. Specifically, the input optical fiber 361 of the delivery channel connects to the rotating carriage drum 325 via an input optical fiber rotary coupling 360. This allows the input optical fiber 361 to remain stationary, i.e., not rotate. In the current implementation, a tunable laser provides the tunable optical signal on the input optical fiber 361. In other applications, a narrowband optical source is used for reflectivity analysis. In other systems, a broad band source is used.

An electrical slip ring system 363 transmits electrical power and signals to and from the rotating drum. Specifically, a spectral analysis system 22 is provided, in one embodiment, to receive spectral data from the slip ring system 363 to enable analysis of the target tissue. A stabilizing bracket 365 prevents the nominally stationary side of the rotary coupling from rotating due to torque transfer through the coupling from the rotating drum 325.

FIG. 7A shows the optical and electrical systems illustrating their relationship to the rotating drum 325.

The delivery tunable optical signal, such as generated by a tunable laser 20, is transmitted on fiber 361, through the input optical fiber rotary coupling 360, to the rotating drum 325. The input optical fiber 361d in the drum 325 connects to a tap 368. This tap 368 directs a portion of the optical signal transmitted by the input optical fiber 361d, the delivery channel, to a delivery signal detector 364 on the drum 325. The remaining signal is transmitted on fiber 361e of the delivery optical fiber 74 of the catheter system 100 via the duplex couplers 312/120. Any collected optical signal collected from the catheter head 110 is transmitted through the collection fiber 72 of the catheter system 100 and received on the collection optical fiber 370 of the collection channel. This optical fiber terminates on a collection optical detector 366.

In general, a delivery channel transmits the optical signals to the intraluminal catheter 100 via the rotating drum 325 through rotary joint 360 and the delivery channel detector 364 on the rotation carriage monitors the optical signals being transmitted on the delivery channel. The collection channel detector 366 detects optical signals from the patient. A noise suppression system uses the delivery channel detector 364 to reduce noise in the optical signals from the patient introduced by the rotary joint 360 and/or laser noise.

Typically, the optical rotary coupler 360 will inject noise. Another source of noise is the laser itself due to temporal fluctuations in optical power output. The tap 368 provides a portion of this delivery optical signal, including any noise to the delivery optical signal detector 364. Then, when the returning optical signal from the catheter head 100 is received and detected by the collection detector 366, the noise added by the rotary coupling 360 and any laser noise is removed by the processing performed by the divider 368. Specifically, the system provides for common mode rejection which will remove noise introduced by the rotary joint 360 and laser noise. Thus, the output optical signal without the noise is then further provided to the spectral analysis engine 22 that resolves the spectral response of the patient tissue that allows for its analysis, for example, determining the state of the tissue. In other examples, OCT analysis is performed to determine the topology of the tissue.

In other embodiments, the delivery optical signal detector is located not on the rotating drum 325 but is between the drum 325 and the laser 20. This is used in situations in which any noise from the rotary coupler 360 is minimal or outside the signal band.

The incorporation of the optical detectors 364, 366 on the rotating drum 325 provides a number of advantages. First, since the collection optical detector 366 is on the drum 325, a second optical rotating coupler is not required. The information in the optical signals is transmitted electrically from the rotating drum 325 via electrical slip ring system 363. One problem that arises when using optical rotating rotary coupling is the potential for the creation of optical noise due to the rotating movement of the coupler 360. This is addressed in the present system by the incorporation of the delivery detector 364 on the rotating drum 325.

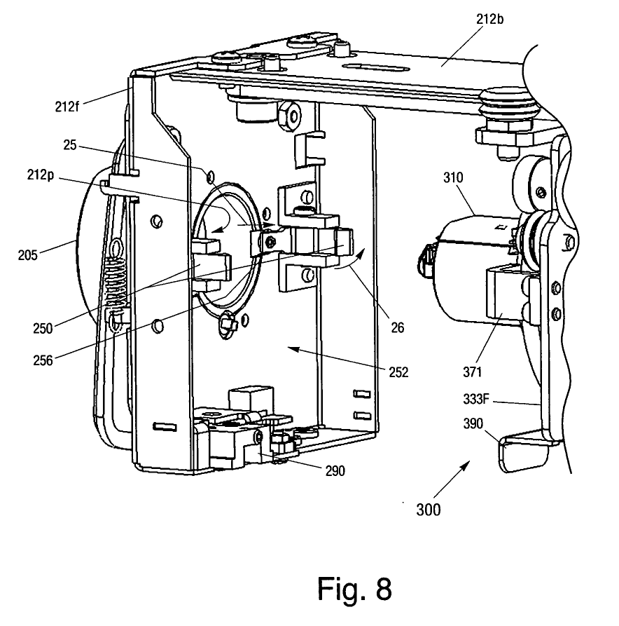

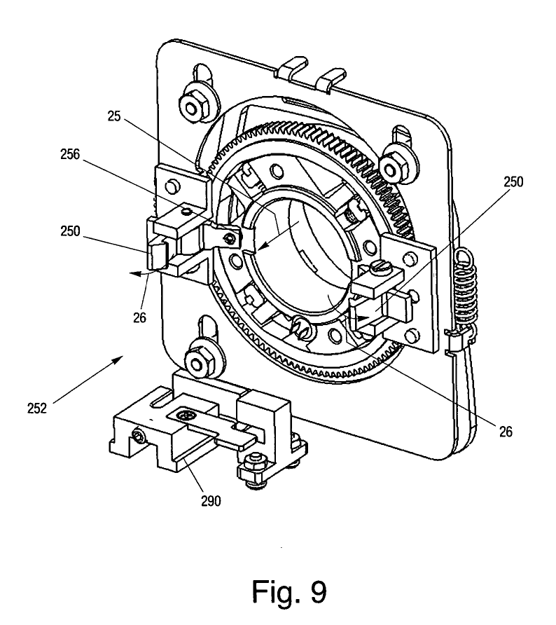

FIGS. 8 and 9 illustrate the carriage drive interlock system. This interlock system ensures that the carriage drive system 300 is and is held at or near the proximal end of the pullback and rotation frame, near the front member 212f especially during the attachment of the catheter system 100 to the pullback and rotation system 200.

In general, the carriage drive interlock is a latching system 252 for holding the carriage drive system 300 of the pullback and rotation system 200 from moving when the catheter system 100 is being attached to the pullback and rotation system 200. It further has a release system for unlocking the latching system 252 to enable longitudinal movement of the carriage drive system 300 relative to the frame 212 upon connection of the catheter system 100 to the pullback and rotation system 200. The interlock latching system 252 ensures that the carriage drive system 300 does not move freely, specifically in response to any attachment force supplied by the operator in order to attach the catheter system 100 to the interface 205 on the pullback and rotation system 200.

Specifically, two carriage latches 250 lock and engage with two opposed carriage latch plates 370 that extend from the front face of the front carriage frame piece 333f. Specifically, each carriage latch 250 engages with a corresponding carriage latch plate 371 (see FIG. 8, for example) to lock the carriage drive system 300 in its forward position. A forward position sensor 290 on the frame 212 detects the presence of flag arm 390 to confirm that the carriage drive system 300 is in its forward most position. In this position, the carriage coupler mount 310 projects thought port 212p in the front member 212f of the frame 212 enabling the carriage 118 of the catheter system 100 to mechanically and optically mate with the carriage drive system 300. Each of the carriage latches 250 is biased into engagement with the plates 371 by a bias spring.

The carriage drive system 300 becomes unlatched only upon full insertion of the catheter system 100 onto the pullback and rotation system 200 through the action of the release system. Specifically, the full insertion and attachment of the carriage system 100 causes the catheter sensing pin 256 to move in the direction of arrow 25. This movement pivots the carriage latches 250 in the direction of arrow 26 to disengage from the carriage latch plates 371, thereby freeing the carriage drive system 300 to move longitudinally on the frame rails 212r.

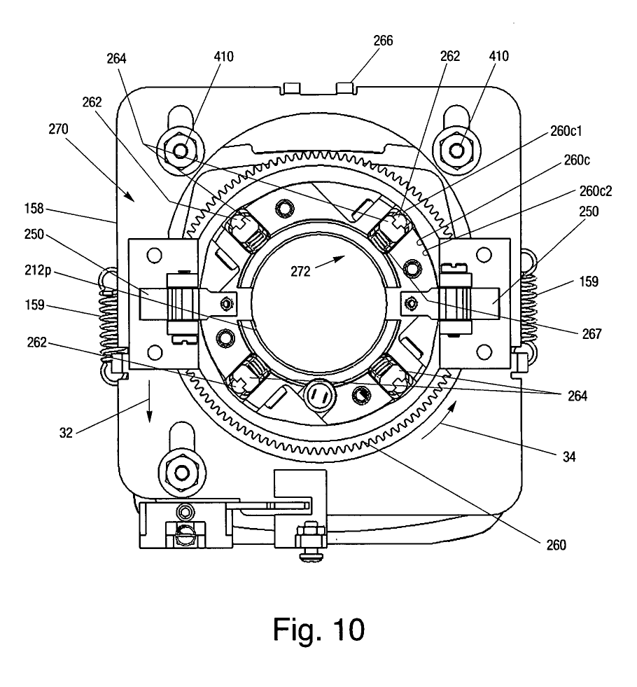

FIG. 10 illustrates a catheter housing interlock system 270 that ensures that the catheter system 100, and specifically the catheter housing 112, is not accidentally disconnected from the pullback and rotation system 200. The catheter housing interlock system 270 includes four catheter housing locking mechanisms 272 for securing the catheter housing 112 to the front frame member 212f of the pullback and rotation system 200.

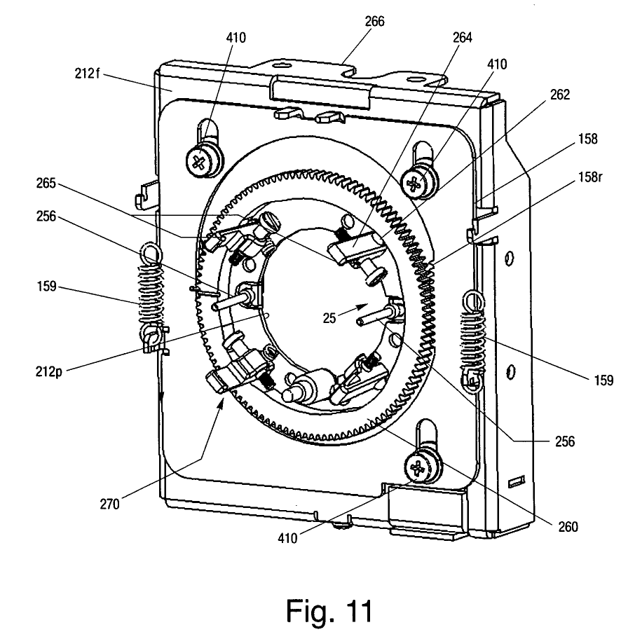

Specifically, the catheter housing interlock system 270 comprises a catheter locking rack frame 158. When this catheter locking rack frame 158 is depressed by the operator in the direction of arrow 32, by applying a downward force on tab 266, it causes the locking cam gear 260 to rotate in the direction of arrow 34. In more detail, guide pin bolts 410 attached to the front member 212f guide the rack frame to slide vertically against the force of bias rack springs 159. A rack gear 158r (see FIG. 11) of the rack frame 158 engages teeth on the outer periphery of the locking cam gear 260. The rotation of the cam gear causes the camming surface 260c on the inner face of the catheter cam gear 260 to engage and push in a radial inward direction the four locking rollers 262 as region 260c1 moves away from roller 262 and region 260c2 comes into contact with rollers 262. This moves the locking rollers 262 and the latches 264 against the spring elements 267.

FIG. 11 shows the front side of the catheter interlock system 270. The interface ring 110 is removed to expose the latches 264 that would normally extend through the ports 210p of the interface ring 210, see FIG. 4. The rotation of the cam gear 260 causes the latches 264 to pivot radially outward with respect to the central port 212p. Please refer to FIG. 11. The pivoting of the latches 264 outward causes the latch shoulders 265 to pull away from the housing locking shoulders 112s of the catheter system 100. Refer to FIG. 3. Thus, only when the operator applies a downward force on tab 266, moving the rack 158 against bias spring 159, will the catheter system 100 become free from the pullback and rotation system 200. This ensures that the catheter housing 112 does not become disconnected from the pullback and rotation system 200 in an uncontrolled fashion against the intent of the operator.

While this invention has been particularly shown and described with references to preferred embodiments thereof, it will be understood by those skilled in the art that various changes in form and details may be made therein without departing from the scope of the invention encompassed by the appended claims.

What is claimed is:

1. A catheter system, comprising:
an intraluminal catheter for insertion into a patient including:
an outer jacket, and
an inner scanning body that rotates and moves longitudinally within the outer jacket; and
a handle portion, including:
an outer housing mechanically coupled to the outer jacket, and
an inner carriage at least partially within the outer housing and mechanically coupled to the inner scanning body.

2. A catheter system as claimed in claim 1, wherein the inner scanning body comprises an optical fiber bundle, including at least one optical fiber, for transmitting optical signals between a head of the intraluminal catheter and the inner carriage.

3. A catheter system as claimed in claim 2, wherein the inner carriage comprises one or more optical connectors for providing optical connection to the optical fiber bundle.

4. A catheter system as claimed in claim 1, wherein the inner scanning body comprises a torque cable for transferring rotation from the inner carriage through the inner scanning body to a head of the intraluminal catheter.

5. A catheter system as claimed in claim 1, wherein the outer housing functions as a handle for attachment of the catheter system to a pullback and rotation system.

6. A catheter system as claimed in claim 1, wherein the inner carriage comprises a bayonet member projecting axially from a proximal side of the inner carriage for rotationally aligning the inner carriage during attachment to a pullback and rotation system.

7. A catheter system as claimed in claim 1, further comprising a catheter carriage interlock system that secures the inner carriage within the outer housing at least during transportation.

8. A catheter system as claimed in claim 7, wherein the catheter carriage interlock system prevents rotation of the inner carriage within the outer housing.

9. A catheter system as claimed in claim 7, wherein the catheter carriage interlock system prevents extraction of the inner carriage from the outer housing.

10. A catheter system as claimed in claim 9, wherein the catheter carriage interlock system prevents rotation of the inner carriage within the outer housing.

11. A catheter system as claimed in claim 7, further comprising a release member on a pullback and rotation system for disengaging the catheter carriage interlock system so that the inner carriage is able to rotate and move relative to the outer housing.

12. A catheter system as claimed in claim 7, wherein the catheter carriage interlock system comprises at least one lever arm that pivots to engage the inner carriage to prevent rotation and/or extraction of the inner carriage from the outer housing.

13. A catheter carriage interlock system for an optical catheter system comprising an intraluminal catheter that provides optical signals to a patient and carries optical signals from the patient, an outer housing, and an inner carriage that moves longitudinally relative to the outer housing and rotates relative to the outer housing during operation when the catheter system is being driven by a pullback and rotation system, the interlock system comprising:
a carriage locking system that prevents rotation and longitudinal movement of the inner carriage in the outer housing; and
an unlocking system on the pullback and rotation system that unlocks the carriage locking system to free the inner carriage to rotate and move longitudinally relative to the outer housing when the catheter system is connected to the pullback and rotation system.

14. An interlock system as claimed in claim 13, wherein the catheter locking system comprises at least one lever arm that pivots to engage the inner carriage to prevent rotation and/or extraction of the inner carriage from the outer housing.

15. An interlock system as claimed in claim 14, wherein the at least one lever arm mechanically interferes with an extraction shoulder on the inner carriage to prevent extraction of the carriage from the outer housing.

16. An interlock system as claimed in claim 14, wherein the at least one lever arm mechanically interferes with a rotation shoulder on the inner carriage to prevent rotation of the carriage in the outer housing.

17. An interlock system as claimed in claim 16, wherein the at least one lever arm mechanically interferes with an extraction shoulder on the inner carriage to prevent extraction of the carriage from the outer housing.

18. An interlock system as claimed in claim 14, wherein the unlocking system comprises a ring that engages the at least one leverage arm to pivot the at least one lever arm out of engagement with the inner carriage.

19. A catheter carriage interlock method for an optical catheter system comprising an intraluminal catheter that provides optical signals to a patient and carries optical signals from the patient, an outer housing, and an inner carriage that moves longitudinally relative to the outer housing and rotates relative to the outer housing during operation when the catheter system is being driven by a pullback and rotation system, the interlock method comprising:
preventing rotation and longitudinal movement of the inner carriage in the outer housing at least during transportation of the optical catheter system; and
unlocking the inner carriage to free the inner carriage to rotate and move longitudinally relative to the outer housing when the optical catheter system is connected to a pullback and rotation system.

20. A catheter system, comprising:
an intraluminal catheter for insertion into a patient including:
an outer jacket, and
an inner scanning body that rotates and moves longitudinally within the outer jacket during operation; and
a handle portion, including an outer housing mechanically coupled to the outer jacket, and
a catheter housing locking mechanism for securing the handle portion to a frame of the pullback and rotation system until unlocked by a user.

21. A catheter system as claimed in claim 20, wherein the catheter housing locking mechanism is actuated by a user to release the handle portion from the frame.
